(19) United States
(12) Patent Application Publication
(10) Pub. No.: US 2008/0275049 A1
(43) Pub. Date: Nov. 6, 2008

(54) Methods and Compositions for Upregulation of GATA Activity

(75) Inventors: John Anastasios Polikandriotis, Tampa, FL (US); Sivaram Pillarisetti, Norcross, GA (US); Ish Khanna, Alpharetta, GA (US); Uday Saxena, Atlanta, GA (US)

Correspondence Address: DORITY & MANNING, P.A., POST OFFICE BOX 1449, GREENVILLE, SC, 29602-1449, US

(73) Assignee: REDDY US THERAPEUTICS, INC., Norcross, GA (US)

(21) Appl. No.: 12/113,426

(22) Filed: May 1, 2008

Related U.S. Application Data
(60) Provisional application No. 60/916,214, filed on May 4, 2007.

(51) Int. Cl. A61K 31/53; C12Q 1/02; A61P 9/10; C12Q 1/66

(52) U.S. Cl. 514/245; 435/29; 435/8

(57) ABSTRACT

A novel method of identifying compounds capable of upregulating GATA activity is disclosed. The method includes providing a sample of cells that express GATA, providing a sample of a candidate compound, contacting the cell sample and the compound sample, and measuring a quantitative indicator of GATA activity within the cell sample after the contacting step. GATA inducers identified by the method and uses therefore to upregulate GATA activity in subjects and to prevent or treat atherosclerosis or diabetes in subjects are also described.

### RELATED APPLICATIONS

The present application claims priority to U.S. Provisional Application Ser. No. 60/916,214, which was filed on May 4, 2007, the contents of which are incorporated herein by reference thereto.

### BACKGROUND OF THE INVENTION

The present invention relates to reverse cholesterol transport. More specifically, the present invention relates to a method of modulating the rate of reverse cholesterol transport and/or reducing or preventing arterial plaque build-up.

Cholesterol is a lipid found in the cell membranes of all body tissues, and is transported in the blood plasma of all animals. Forms of cholesterol include low-density lipoprotein (LDL), often referred to as “bad cholesterol” and high-density lipoprotein (HDL), often referred to as “good cholesterol.”

Cholesterol is required to build and maintain cell membranes. Cholesterol also aids in the manufacture of fat soluble vitamins, including vitamins A, D, E, and K. It is the major precursor for the synthesis of Vitamin D and of the various steroid hormones.

Elevated levels of LDL are regarded as atherogenic, or prone to cause atherosclerosis. Atherosclerosis is a disease affecting the arterial blood vessels. It is a chronic inflammatory response in the walls of the arteries, in large part due to the deposition of lipoproteins in the form of plaque. It is commonly referred to as “hardening” of the arteries. Studies have shown that high levels of LDL contribute to the formation of plaque in the arteries, while high levels of HDL prevent formation of plaque or decrease previously formed plaque in the arteries.

Statins (or HMG-CoA reductase inhibitors) form a class of hypolipidemic agents, used as pharmaceutical agents to lower cholesterol levels in people with or at risk for cardiovascular disease. They lower cholesterol by inhibiting the enzyme HMG-CoA reductase, which is the rate-limiting enzyme of the mevalonate pathway of cholesterol synthesis. Inhibition of this enzyme in the liver stimulates LDL receptors, resulting in an increased clearance of LDL from the bloodstream and a decrease in blood cholesterol levels. The first results can be seen after one week of use and the effect is maximal after four to six weeks. Although statins are effective at lowering LDL levels, they have not demonstrated an ability to increase HDL levels.

Despite their ability to lower LDL levels in the bloodstream, statins have been shown to be only minimally effective at reducing or preventing plaque build-up in the arteries that may lead to atherosclerosis.

Studies show that elevated levels of HDL are effective at reducing and preventing plaque build-up. A reduction in plaque build-up is an effective treatment for atherosclerosis. It is believed that the prevention of plaque build-up would effectively prevent atherosclerosis.

Cholesterol that is synthesized or deposited in peripheral tissues, such as macrophages within the arterial wall, is returned to the liver in a process referred to as reverse cholesterol transport (RCT) in which HDL plays a central role and therefore is believed to protect against atherosclerosis. HDL may be secreted by the liver or intestine. HDL interacts with peripheral cells, such as macrophages, to facilitate the removal of excess free cholesterol (FC), a process facilitated by the ATP-binding cassette protein 1 (ABCA1) gene. HDL is then converted into mature FC-rich HDL as a result of the plasma cholesterol-esterifying enzyme lecithin:cholesterol acyltransferase (LCAT). FC may then be removed by selective uptake by the liver, i.e., the removal of lipid without the uptake of HDL proteins. Selective uptake appears to be mediated by the scavenger receptor class-B, type I (SRB1), which is expressed in the liver and has been shown to be a receptor for HDL. FC derived from HDL contributes to the hepatic-cholesterol pool used for bile acid synthesis. Cholesterol is eventually excreted from the body either as bile acid or as free cholesterol in the bile. Ultimately, promotion of the RCT pathway could help prevent or reduce atherosclerosis.

An increase in the activity of ABCA1, LCAT, and SRB1, typically results in a boost in HDL levels and in the reduction or prevention of plaque in the arteries. The ABCA1, LCAT, and SRB1 genes are, therefore, commonly referred to as reverse cholesterol transport genes.

Elevated levels of LDL and triglycerides and low levels of HDL are often found in diabetics and this phenotype is referred to as “diabetic dyslipidemia”. This condition results in cholesterol accumulation in tissues that are important in glucose metabolism. Cholesterol accumulation in tissues may lead tissue dysfunction, for example cholesterol accumulation in pancreas may result in decreased secretion of insulin, the critical hormone required for glucose uptake. Cholesterol accumulation on other tissues e.g. adipose and skeletal muscle may lead to insulin resistance thus defective glucose uptake in response to insulin. Removal of cholesterol from these tissue will have a beneficial effect on insulin resistance, pancreatic function and thus, for prevention and treatment of diabetes.

GATA is a family of transcription factors involved in cell growth. These factors are essential for erythroid (red blood cell) and megakaryocytic development. Mice without GATA die as embryos. Each GATA molecule contains three domains, the C-finger, the N-finger, and the Activation Domain. The C-finger, named for being near the C-terminal, has two highly conserved Zinc finger binding domains, which form the Activation Domain that binds the consensus sequence (A/T)GATA(A/G). The N-finger, named for being near the N-terminal also binds DNA and a cofactor named FOG-1. The Activation Domain is responsible for GATA's strong transcriptional activation. The gene for GATA is on the X-chromosome.

Data presented herein show that GATA factor is an important component in the activation of RCT pathway genes and proteins. It would be desirable, therefore, to develop a screening method to identify compounds that are GATA activators. These compounds could then be utilized to activate GATA, thereby increasing the activity of ACBA1, LCAT, and SRB1 and the rate of reverse cholesterol transport. Compounds that are identified by this method as being GATA activators would also be useful for therapeutic applications such as the prevention and/or treatment of diseases associated with cholesterol formation, deposition and transport such as atherosclerosis and diabetes.

### SUMMARY OF THE INVENTION

Briefly, therefore the present invention is directed to a novel method of identifying compounds capable of upregulating GATA activity. The method includes proving a sample of cells that express GATA, providing a candidate GATA activity-modulating compound, contacting the cell sample and the GATA activity-modulating compound sample in the presence of an assay for GATA activity, and measuring the change in GATA activity within the cells.

The utility of GATA activating compounds in the treatment of atherosclerosis and diabetes is described. These and other aspects of the invention will be understood and become apparent upon review of the specification by those having ordinary skill in the art.

### BRIEF DESCRIPTION OF THE DRAWINGS

FIG. 1 illustrates the experimental results of Example 1.

FIG. 2 illustrates the experimental results of Example 2.

FIG. 3 illustrates the experimental results of Example 3.

FIG. 4. illustrates the experimental results of Example 4.

FIG. 5 illustrates the experimental results of Example 5.

FIG. 6 illustrates the experimental results of Example 6.

FIG. 7 illustrates the experimental results of Example 7.

FIG. 8 illustrates the experimental results of Example 8.

FIG. 9 illustrates the experimental results of Example 9.

### DETAILED DESCRIPTION OF THE PREFERRED EMBODIMENTS

Reference now will be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used on another embodiment to yield a still further embodiment. Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

In one aspect, the invention is a method of identifying compounds capable of upregulating GATA activity. The method includes proving a sample of cells that express GATA, providing a sample of a GATA activity-modulating candidate compound (a “candidate compound”), contacting the cell sample and the compound sample in the presence of an assay for GATA activity, and measuring the change in GATA activity that is caused by the contact with the candidate compound. It has been found to be useful to use a high-throughput assay based on luciferase activity for the assay for GATA activity as the measure of a quantitative indicator of the change in GATA activity within the cell sample that is caused by the candidate compound.

The GATA family of transcription factors is known to include GATA1, GATA2, GATA3, GATA4, GATA5, and GATA6. As used herein, the term GATA shall be interpreted as including one or more of the GATA family of transcription factors unless explicitly stated otherwise.

In a preferred embodiment, the present invention is a method of identifying compounds capable of upregulating GATA1 activity.

In one embodiment, the cells that express GATA are human liver cells.

In preferred embodiments, the quantitative indicator of GATA activity is luciferase activity. In one embodiment, the step of contacting the cell sample and the candidate sample in the presence of a high-throughput assay based on luciferase activity includes contacting the cell sample and the candidate sample in a plasma in which the GATA transcription binding domain is bound to the luciferase assay. When the sample candidate successfully upregulates the GATA activity, luciferase is freed from the binding domain and produces light. As GATA activity increases, the luciferase activity increases, resulting in a similar increase in free luciferase in the assay. In these embodiments, the quantitative activity that is measured is the light given off by the freed luciferase.

The quantitative indicator may also be one or more post-translational activities, including, but not limited to phosphorylation, localization, or acetylation.

In the present screening method, an increase in the monitored quantitative indicator indicates an upregulation of GATA activity. As used herein, the terms “GATA activity” refer to the amount of or concentration of a GATA transcription factor and/or the activity of a GATA transcription factor in the modulation of the RCT pathway. Accordingly, in the present method, when the monitored quantitative indicator indicates an increase in GATA activity upon contact of the cell sample with the candidate compound, the candidate compound is shown to be effective in upregulating the GATA activity.

It has been shown that, surprisingly, an upregulation in GATA activity results in an increase in the activity of the RCT genes (ABCA1, LCAT, SRB1 and ApoE). As is known to those having ordinary skill in the art, an increase in the activity of the RCT genes serves to raise HDL levels and reduce and/or plaque build-up arteries. In one aspect, therefore, the invention is a method of increasing the activity of the RCT genes, and thereby increasing RCT, raising levels of HDL and preventing and/or reducing plaque build-up in arteries.

In another aspect, the invention is a method of increasing the activity of the RCT genes, and thereby increasing RCT, and for prevention and/or treatment of diabetes and insulin resistance. In another aspect this invention is a method for improving pancreatic beta cell function.

In another aspect, the invention is a method of increasing the rate of reverse cholesterol transport. The method includes administering to a subject in need of an increased rate of reverse cholesterol transport an effective amount of a GATA inducer to increase the rate of reverse cholesterol transport. As used herein, the term “GATA inducer” will be understood by those having ordinary skill in the art as including any compound that increases GATA activity. By way of example, any compound that causes an increase in GATA activity in the present method of identifying GATA inducers that is described herein, is considered to be a GATA inducer.

In some embodiments, GATA inducers may be selected from the group consisting of:

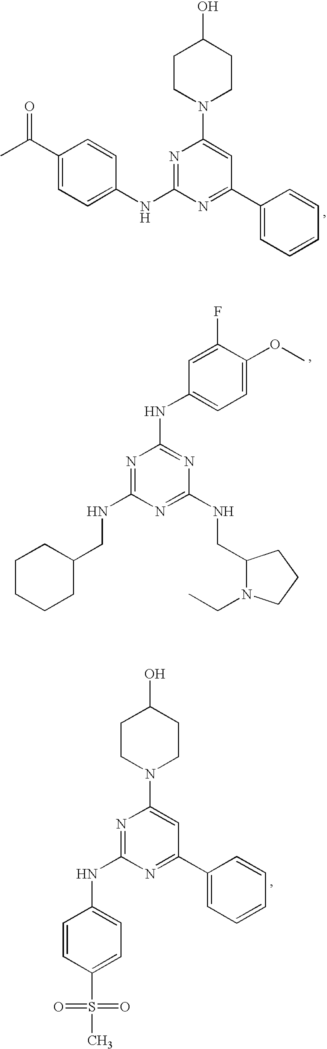

and isomers, tautomers, salts of these compounds, and combinations thereof.

For ease of reference, the present invention will be described with reference to administration to human subjects. It will be understood, however, that such descriptions are not limited to administration to humans, but will also include administration to other animals, such as mammals, unless explicitly stated otherwise.

The present method includes administering one or more GATA inducer to the subject by administration means known in the art. Administration means contemplated as useful include one or more of topically, buccally, intranasally, orally, intravenously, intramuscularly, sublingually, and subcutaneously. Other administration means known in the art are also contemplated as useful in accordance with the present invention and are discussed in more detail below.

In some embodiments, it may be useful to include one or more of the GATA inducers as a salt. Those having ordinary skill in the art will recognize the salts of the GATA inducer compounds.

In some embodiments, the composition may be an aqueous composition. The composition may also be nebulized or aerosolized.

The subject invention involves the use of a safe and effective amount of one or more GATA inducers for increasing the rate of Reverse Cholesterol Transfer, thereby treating or preventing atherosclerosis and other conditions caused by low levels of HDL. Subjects in need of an increase in the rate of RCT include, but are not limited to, subjects having low levels of HDL, subjects having plaque-build-up in arteries, subjects suffering from atherosclerosis, and subjects in need of prevention of atherosclerosis.

An exemplary method of administering one or more GATA inducers is topical, intranasal administration, e.g., with nose drops, nasal spray, or nasal mist inhalation. Other exemplary methods of administration include one or more of topical, bronchial administration by inhalation of vapor and/or mist or powder, orally, intravenously, intramuscularly, and subcutaneously.

Other ingredients which may be incorporated in the present invention include safe and effective amounts of preservatives, e.g., benzalkonium chloride, thimerosal, phenylmercuric acetate; and acidulants, e.g., acetic acid, citric acid, lactic acid, and tartaric acid. The present invention may also include safe and effective amounts of isotonicity agents, e.g., salts, such as sodium chloride, and more preferably non-electrolyte isotonicity agents such as sorbitol, mannitol, and lower molecular weight polyethylene glycol.

In the present method, a subject in need of increased RCT rates is treated with an amount of one or more GATA inducers, where the amount of the one or more GATA inducers provides a dosage or amount that is sufficient to constitute a treatment or prevention effective amount.

As used herein, an “effective amount” means the dose or amount of a GATA inducer to be administered to a subject and the frequency of administration to the subject which is readily determined by one of ordinary skill in the art, by the use of known techniques and by observing results obtained under analogous circumstances and has some therapeutic action. The dose or effective amount to be administered to a subject and the frequency of administration to the subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of the subject to be treated, and other relevant circumstances.

The phrase “therapeutically-effective” indicates the capability of an agent to prevent, or improve the severity of, the disorder, while avoiding adverse side effects typically associated with alternative therapies.

The one or more GATA inducers can be supplied in the form of a novel therapeutic composition that is believed to be within the scope of the present invention.

When the one or more GATA inducers are supplied along with a pharmaceutically acceptable carrier, a pharmaceutical composition is formed. A pharmaceutical composition of the present invention is directed to a composition suitable for the prevention or treatment of the disorders described herein. The pharmaceutical composition comprises at least a pharmaceutically acceptable carrier and one or more GATA inducers. Pharmaceutically acceptable carriers include, but are not limited to, physiological saline, Ringer's, phosphate solution or buffer, buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective.

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included in present invention are the isomeric forms and tautomers and the pharmaceutically-acceptable salts of GATA inducers. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

Suitable pharmaceutically-acceptable base addition salts of compounds of the present invention include metallic ion salts and organic ion salts. More preferred metallic ion salts include, but are not limited to, appropriate alkali metal (Group IA) salts, alkaline earth metal (Group IIA) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

The terms “treating” or “to treat” means to alleviate symptoms, eliminate the causation either on a temporary or permanent basis, or to prevent or slow the appearance of symptoms. The term “treatment” includes alleviation, elimination of causation of or prevention of any of the diseases or disorders described above. Besides being useful for human treatment, these combinations are also useful for treatment of mammals, including horses, dogs, cats, rats, mice, sheep, pigs, etc.

The term “subject” for purposes of this application includes any animal. The animal is typically a human. A preferred subject is one in need of treatment or prevention of the disorders discussed herein.

For methods of prevention, the subject is any human or animal subject, and preferably is a subject that is in need of prevention and/or treatment of atherosclerosis or other disorders caused by low levels of HDL and/or high levels of LDL. The subject may be a human subject who is at risk of disorders such as those described above. The subject may be at risk due to genetic predisposition, sedentary lifestyle, diet, exposure to disorder-causing agents, exposure to pathogenic agents and the like.

The present pharmaceutical compositions may be administered enterally and/or parenterally. Parenteral administration includes subcutaneous, intramuscular, intradermal, intramammary, intravenous, and other administrative methods known in the art. Enteral administration includes solution, tablets, sustained release capsules, enteric coated capsules, syrups, beverages, foods, and other nutritional supplements. When administered, the present pharmaceutical composition may be at or near body temperature.

The phrase “therapeutically-effective” and “effective for the treatment, prevention, or inhibition,” are intended to qualify the amount of each agent for use in the therapy which will achieve the goal of increased proteoglycan levels, while avoiding adverse side effects typically associated with alternative therapies.

In particular, the GATA inducers of the present invention, or compositions in which they are included, can be administered orally, for example, as tablets, coated tablets, dragees, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, any of a variety of herbal extracts, milk, or olive oil.

Aqueous suspensions can be produced that contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing one or more GATA inducers may be formulated with sweetening agents, for example glycerol, sorbitol or sucrose. Such formulations may also contain a demulcent, a preservative, and flavoring and coloring agents.

The subject GATA inducers and compositions in which they are included can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing or wetting agents and suspending agents which have been mentioned above, or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, n-3 polyunsaturated fatty acids may find use in the preparation of injectables.

The subject GATA inducers and compositions in which they are included can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

The subject GATA inducers and compositions in which they are included can also be administered topically, in the form of creams, ointments, jellies, collyriums, solutions, patches, or suspensions.

Daily dosages of the GATA inducers can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages.

Various delivery systems in addition to nutritional supplements include sprays, capsules, tablets, drops, and gelatin capsules, for example.

Those skilled in the art will appreciate that dosages for the therapeutic use of the GATA inducers may also be determined with guidance from Goodman & Goldman's The Pharmacological Basis of Therapeutics, Ninth Edition (1996), Appendix II, pp. 1707-1711.

Preferred dosages for the GATA inducers are those that are effective to increase the rate of RCT. In especially preferred embodiments, the dosage should be in a concentration effective to increase the rate of RCT such that plaque build-up in the arteries is reduced. In yet another embodiment an effective dosage is an amount that is effective to increase HDL levels in the subject. In another embodiment, an effective dosage is an amount that is effective to upregulate GATA activity in the subject.

### General Methods

PT-PCR Screen: Human liver cells (HepG2) were grown in T-75 flasks prior to study. 96-well plates were seeded, and the following day, cells were washed with PBS and treated with serum free media for 24 hours. The next day, cells were treated with vehicle (DMSO) or with one of Compounds A-E overnight. RNA was isolated and RT-PCR was performed to examine changes in RCT gene message levels normalized to actin.

GATA activity: HepG2 cells were grown in T-75 flasks prior to study. 24-well plates were seeded in normal medium without antibiotics to yield ˜50% confluency. The following day, cells were transfected according to the manufacturer's protocol and allowed to incubate overnight. The next day, the media was replaced and cells were treated with vehicle (DMSO) or one of Compounds A-E overnight. Cells were lysed by adding lysis buffer and assayed for luciferase activity.

GATA knock-down: HepG2 cells were grown in T-75 flasks prior to study. Transfection of GATA1 specific siRNA in 24-well plates was performed according to the manufacturer's instructions. After 48 hours, RNA was isolated and RT-PCR was performed to examine changes in GATA1 and RCT message levels all normalized to actin. In some experiments, after 48 hours post transfection, media was removed and normal growth media+one of Compounds A-E or DMSO control was added to the wells. RNA isolation and RT-PCR was then performed as stated above.

### Animal Experiments:

Lipid Measurements: Lipid levels were measured at the end of the studies using commercially available kits.

Lipid Content: 8 week old ApoE −/− mice were fed a high fat diet for 2 months and then placed on normal chow. The mice were then orally dosed for 7 days with either CMC-tween (control) or Compound C at 50 mg/kg/day. At the end of the study, the aorta was dissected out, fixed in the O.C.T., sections were prepared, stained with oil-red O, and mounted under cover slips. The oil-red O staining was analyzed using quantitation software and reported as lipid content per lesion area.

Glucose Measurements: Glucose levels were measured at the end of the studies using commercially available kits.

### EXAMPLE 1

This illustrates the role of GATA in RCT Gene Induction:

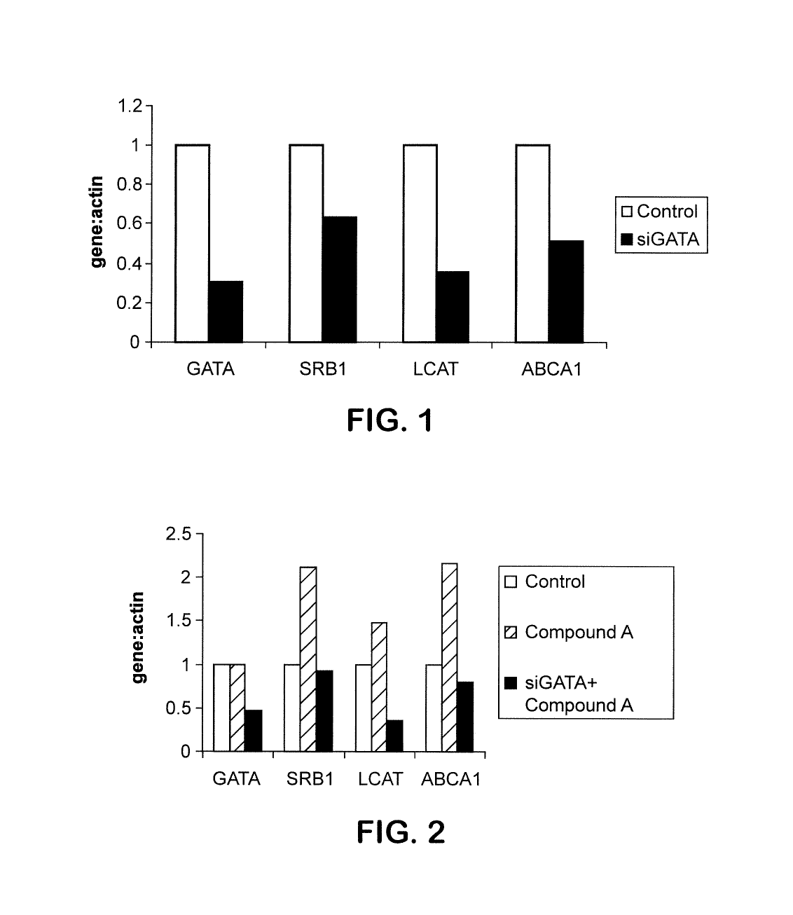

To determine the role of GATA on basal RCT gene expression, molecular approaches were used to reduce GATA expression (GATA knockdown). GATA expression was reduced by siRNA gene silencing. Knocking down GATA expression resulted in reduced levels of basal RCT gene expression, as demonstrated in FIG. 1. The y-axis in FIG. 1 represents the RCT gene activity. As can be seen, the control samples, in which GATA knockdown has not occurred, maintained higher gene activity than the samples in which GATA knockdown was conducted.

### EXAMPLE 2

This illustrates the role of GATA on inducible RCT gene expression:

To determine the role of GATA on inducible RCT gene expression, molecular approaches were used to reduce GATA expression. GATA expression was reduced by siRNA gene silencing. In this test, a candidate compound (Compound A), known for its ability to function as a GATA inducer, was added to both normal and GATA-knockdown cells as described above. FIG. 2 demonstrates that knocking down GATA expression resulted in blunting of Compound A's ability to induce RCT gene expression.

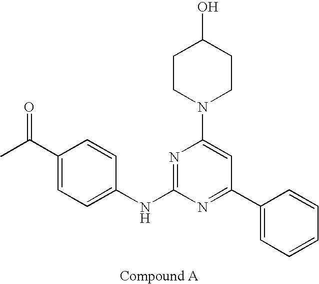

### Compound A

### EXAMPLE 3

This illustrates the identification of GATA inducers.

An in vitro high-throughput assay based on luciferase activity to screen for and identify agents that upregulate GATA activity was developed. HepG2 cells were grown in T-75 flasks prior to study. 24-well plates were seeded in normal medium without antibiotics to yield ˜50% confluency. The following day, cells were transfected (Roche, FuGENE 6 transfection reagent, cat#11815091001) with a GATA luciferase construct (Panomics, cat#LR0027) according to the manufacturer's protocol and allowed to incubate overnight. The next day, the media was replaced and cells were treated with vehicle (DMSO) or GATA inducer candidate compounds (Compounds A-E) overnight. Cells were lysed by adding lysis buffer (Promega, cat#E1500) and assayed for luciferase activity (Perkin Elmer Envision 2101 Multi-Label Reader).

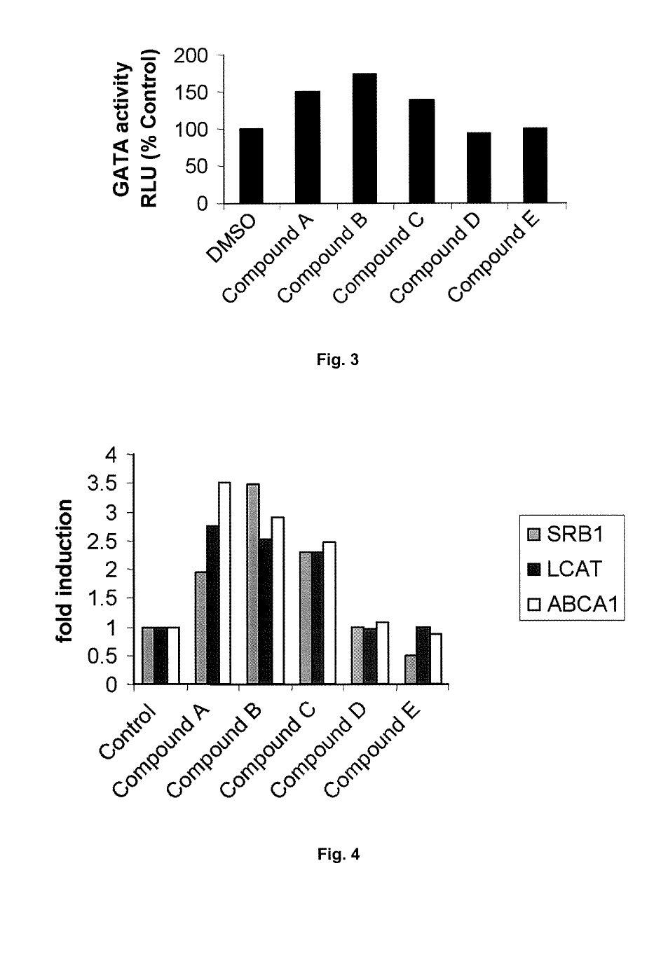

As seen in FIG. 3, Compounds A-C were shown to induce GATA activity, and Compounds D and E were not shown to induce GATA activity.

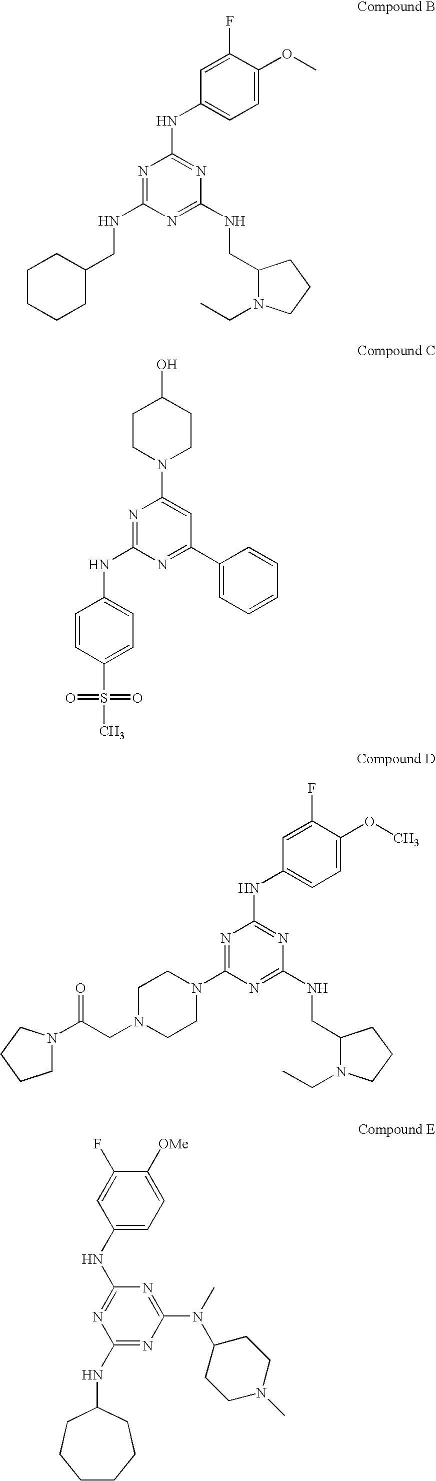

### EXAMPLE 4

This illustrates the therapeutic use of GATA inducers on RCT gene induction.

An in vitro high-throughput assay based on real time quantitative PCR was developed to screen for and identify agents that simultaneously upregulate RCT genes. Cells were treated with pharmaceutical agents, some of which are GATA inducers as described above. Total RNA was isolated and used in the real time assay. Compounds A-C, which were shown to induce GATA activity in Example 3, were shown to induce RCT gene expression, while Compounds D and E, which were not shown to induce GATA activity in Example 3 did not induce RCT gene induction. These results are shown in FIG. 4.

### EXAMPLE 5

This illustrates the therapeutic use of GATA activity and RCT induction in lipid modulation.

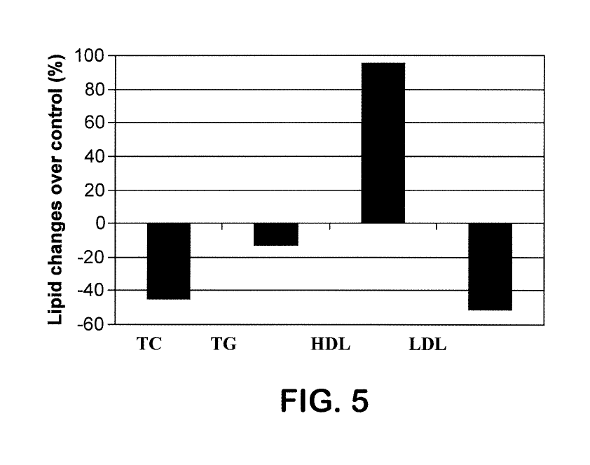

Atherosclerosis is associated with dyslipidemia (increased LDL and low HDL). Administering Compound A to an animal cholesterol fed rat mode significantly increased HDL, as seen in FIG. 5.

### EXAMPLE 6

This illustrates the efficacy of a GATA inducer in reducing the lipid content of aortas in mice.

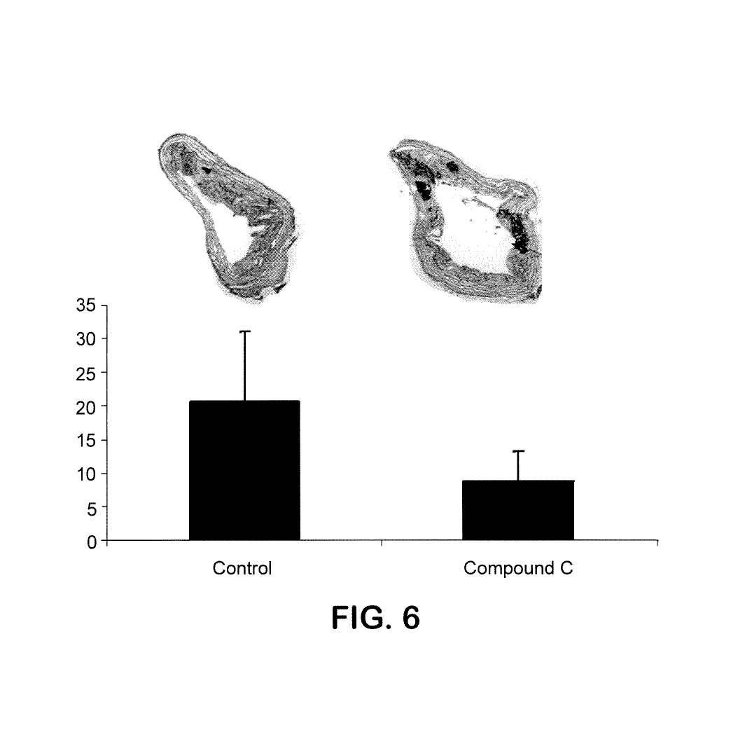

Atherosclerosis is also associated with high levels of lipid content in aortas. Compound C was administered to mice according to the method described above and according to conventional animal testing protocol. The lipid content of the aortas was measured. As seen in FIG. 6, administering Compound C in ApoE −/− fat fed mice significantly lowered lipid content in mice aortas.

### EXAMPLE 7

This illustrates the therapeutic use of GATA activity and RCT induction in pancreatic beta cells.

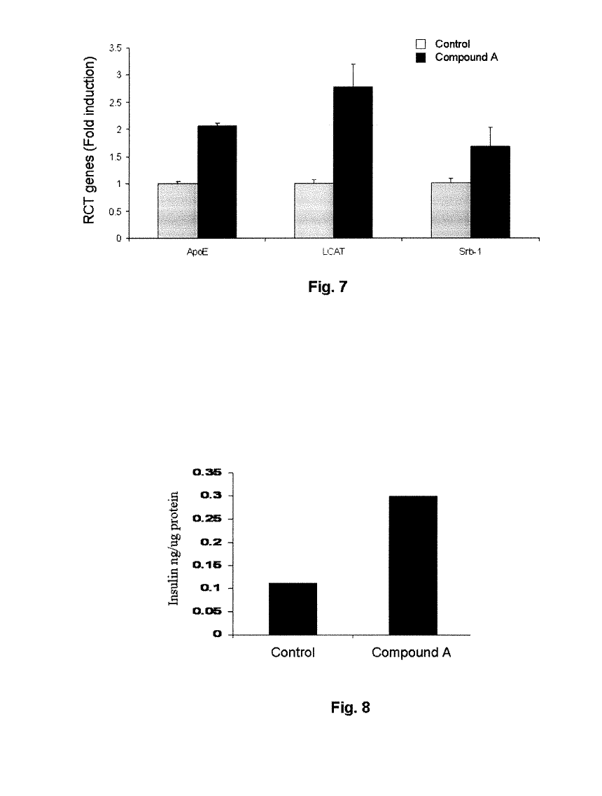

Pancreatic beta cells were incubated with compound A (25 uM). Following treatment total RNA was isolated and RCT gene (apolipoprotein E, SRB1 and LCAT) expression was determined. Treatment with Compound A resulted in an increased expression of RCT genes as seen in FIG. 7.

### EXAMPLE 8

This illustrates the efficacy of a GATA inducer in promoting insulin secretion in pancreatic beta cells.

Pancreatic beta cells were incubated with compound A (25 uM). Following treatment glucose stimulated insulin secretion was determined. Treatment with Compound A resulted in an increased secretion of insulin as seen in FIG. 8.

### EXAMPLE 9

This illustrates the efficacy of a GATA inducer in an animal model of insulin resistance and diabetes.

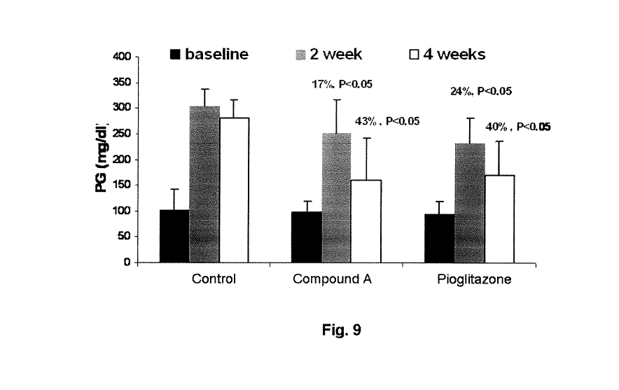

Ob/ob mice were treated with control vehicle or vehicle containing Compound A (GATA activator) or vehicle containing pioglitazone (a known antidiabetic drug). Following 2 and 4 weeks of treatment animals treated with Compound A had significantly lower levels of plasma glucose. The glucose lowering activity of Compound A was similar to pioglitazone as seen in FIG. 9.

In view of the above, it will be seen that the several advantages of the invention are achieved and other advantageous results obtained.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

As various changes could be made in the above methods and compositions by those of ordinary skill in the art without departing from the scope of the invention, it is intended that all matter contained in the above description and shown in the accompanying drawings shall be interpreted as illustrative and not in a limiting sense. In addition it should be understood that aspects of the various embodiments may be interchanged both in whole or in part.

What is claimed is:

1. A method of identifying compounds capable of upregulating GATA activity, the method comprising:
providing a sample of cells that express GATA;
providing a sample of a candidate compound;
contacting the cell sample and the candidate compound; and
measuring GATA activity within the cell sample after the contacting step.

2. The method according to claim 1, wherein the step of measuring GATA activity comprises measuring a quantitative indicator of GATA activity.

3. The method according to claim 1, wherein the cell sample is contacted with the candidate sample in the presence of a high-throughput GATA activity assay based on luciferase activity.

4. The method according to claim 1, wherein the cells that express GATA are human liver cells.

5. The method according to claim 1, wherein the contacting step is in vitro.

6. The method according to claim 2, wherein the quantitative indicator of GATA activity is luciferase activity.

7. The method according to claim 2, wherein the quantitative indicator of GATA activity comprises the measurement of post-translational GATA activity.

8. The method according to claim 7, wherein the post-translational activity is one or more of phosphorylation, localization, or acetylation.

9. A method of increasing the rate of reverse cholesterol transport, the method comprising:
administering to a subject in need of an increased rate of reverse cholesterol transport a compound identified as increasing the activity of GATA.

10. The method according to claim 9, wherein the subject is a human subject.

11. The method according to claim 9, wherein the subject suffers from atherosclerosis.

12. The method according to claim 9, wherein the subject is one in need of prevention of atherosclerosis.

13. The method according to claim 9, wherein the subject suffers from diabetes.

13. The method according to claim 9, wherein the subject suffers from diabetes.

14. The method according to claim 9, wherein the subject is one in need of prevention of diabetes.

15. The method according to claim 9, wherein the administration is at concentrations sufficient to increase HDL concentration in the subject.

16. The method according to claim 9, wherein the administration is at concentrations sufficient to decrease plasma glucose levels in the subject.

17. The method according to claim 9, wherein the administration is at concentrations sufficient to upregulate GATA activity in the subject.

18. A method of treating or preventing atherosclerosis, the method comprising administering to a subject in need of prevention or treatment of atherosclerosis a GATA inducer identified by a method that comprises:
providing a sample of cells that express GATA;
providing a sample of a candidate compound;
contacting the cell sample and the candidate compound; and
measuring GATA activity within the cell sample after the contacting step.

19. A method of treating or preventing diabetes, the method comprising administering to a subject in need of prevention or treatment of diabetes a GATA inducer identified by a method that comprises:
providing a sample of cells that express GATA;
providing a sample of a candidate compound;
contacting the cell sample and the candidate compound; and
measuring GATA activity within the cell sample after the contacting step.

20. A method of increasing the rate of reverse cholesterol transport, the method comprising administering to a subject in need of an increased rate of reverse cholesterol transport a GATA inducer identified by a method comprising:
providing a sample of cells that express GATA;
providing a sample of a candidate compound;
contacting the cell sample and the candidate compound; and
measuring GATA activity within the cell sample after the contacting step.

21. A GATA inducer identified by a method that comprises:
providing a sample of cells that express GATA;
measuring GATA activity within the cell sample after the contacting step.

22. A method of identifying compounds that increase the rate of reverse cholesterol transport, the method comprising:
providing a sample of cells that express GATA;
providing a sample of a candidate compound;
contacting the cell sample and the candidate compound;
measuring GATA activity within the cell sample after the contacting step;
and selecting those candidate compounds that increase GATA activity as compounds that increase the rate of reverse cholesterol transport.
